(19) United States
(12) Patent Application Publication
(10) Pub. No.: US 2026/0083149 A1
(43) Pub. Date: Mar. 26, 2026

(54) Coffee Fermentation Using Pichia Kluyveri and Lactiplantibacillus Plantarum

(71) Applicant: CHR. HANSEN A/S, Hoersholm (DK)

(72) Inventors: Emil ERIKSSON, Hoersholm (DK); Sofie SAERENS, Hoersholm (DK)

(73) Assignee: CHR. HANSEN A/S, Hoersholm (DK)

(21) Appl. No.: 19/112,776

(22) PCT Filed: Sep. 21, 2023
(86) PCT No.: PCT/EP2023/076091

§ 371 Date: Mar. 18, 2025

(30) Foreign Application Priority Data
Sep. 27, 2022 (EP) 22198024.6

(51) Int. Cl. A23F 5/02; C12N 1/16; C12N 1/20; C12R 1/25; C12R 1/84

(52) U.S. Cl. CPC A23F 5/02; C12N 1/165; C12N 1/205; C12R 2001/25; C12R 2001/84

(57) ABSTRACT

The present invention is in the field of food technology. It relates to methods for producing coffee beans, coffee beverages and coffee-based products, characterized in that at fermentation is carried out by inoculating coffee cherries with Pichia kluyveri and Lactiplantibacillus plantarum. The invention provides starter cultures and uses thereof for fermenting coffee plant material, as well as fermented coffee beans prepared according to methods described herein.

### FIELD OF THE INVENTION

The present invention relates to the field of coffee fermentation. In particular, the invention relates to methods of coffee fermentation with microorganisms and coffee products obtained therefrom.

### BACKGROUND OF THE INVENTION

Coffee is a brewed beverage with a distinct aroma and flavor, prepared from the roasted seeds/beans of the Coffea plant, a member of the Rubiaceae family.

Several species of Coffea may be grown for the harvesting of beans. Coffea arabica accounts for 75-80 percent of the world's coffee production, while Coffea canephora accounts for about 20 percent.

When the coffee fruit is ripe, it is often handpicked, using either “selective picking” (where only the ripe fruit is removed) or “strip-picking” (where all fruits are removed from a branch).

Because a tree can have both ripe and unripe berries at the same time, one area of crop has to be picked several times, making harvesting the most labor-intensive process of coffee bean production. Machine harvesting can also be applied, often seen in countries where labor is scarce and/or expensive. When deciding which harvest method to use it is a matter of cost-benefit evaluation.

Coffee fruits can be processed by various methods, including wet-, dry- and semidry-processing. The treatment of the plant material (such as washing, sorting and pulping) and length of time needed, varies depending on the processing type.

Wet processing: The ripe coffee fruits go through a flotation process to clean debris and remove floaters. The coffee fruit is then pulped, in some cases put through an underwater tank fermentation process, and then washed and dried in e.g. the sun, or, in the case of commodity sub-specialty coffee manufacturers, in drying machines. The process is also called Washed process.

Dry processing: The entire coffee fruits are dried in the sun on platforms and/or on the floor without prior removal of the pulp. In some cases, the coffee fruits are cleaned, and floaters are separated immediately after harvesting. In other cases, the sorting is performed after drying. Dry processing is applied on most of the worldwide total coffee production. The process is also called “natural processing”

Semidry processing: The fruit skin is removed within 24 hours of harvest and mucilage is left on the seed while the coffee dry. The process is also called Honey Process or Pulped Natural process.

To meet an increasing demand for unique and high specialty coffee, alternative methods are gaining popularity. An example hereof is variations of pre-treatment, where the freshly harvested coffee fruits are fermented in a fermentation vessel (tank, barrel, bin, sachet) before it goes into the traditional flow of Washed, Natural or Pulped Natural processes. The pre-treatment can be performed with or without sorting, pulping, water addition and/or CO₂ addition. Popular names of pre-treated coffee include Carbonic Maceration (CM Process) and Anaerobic fermentation/process/natural/pulped natural.

After processing has taken place, the green coffee beans are obtained which may be stored and/or shipped.

Spontaneous fermentation in traditional methods occurs without the addition of starter culture. However, the spontaneous fermentation is very inhomogeneous and large variations occur in both microbial counts and species composition, even in fermentations occurring at the same site. The variations depend on many factors including country, farm, weather events, ripeness, post-harvest handling, type of coffee, fermentation method, size of the batch, turning or no turning, fermentation time etc. which makes it difficult to control the coffee fermentations and as a result the reproducibility of the fermentations is almost impossible. A major challenge is the lack of control over the growth and development of microorganisms and metabolites produced during the fermentation process, which results in a variable quality of the finished coffee beans.

Attempts have been made in the prior art to control the fermentation conditions of coffee bean fermentations. WO2014177666 discloses using Pichia yeast strains for fermentation of coffee berries. It discloses that Pichia kluyveri is more preferable than Pichia anomala for fermentation. However, no use of Lactiplantibacillus plantarum was mentioned.

The fermentation process should be controlled to ensure the development of beneficial microorganisms that produce a high-quality beverage with a good aroma. When fermentation fails, it results in the development of spoilage microorganisms that adversely affect the coffee's aroma and flavor.

Thus, being able to regulate the flavor in coffee beans, in combination with high quality beans, would allow for the development of new technologies permitting the coffee producers to obtain high quality coffee beans. Such a technology would, therefore, be of significant commercial value.

Thus, there exists a constant need for improved processes for improvement of the quality of the coffee beans and coffee products prepared therefrom.

### SUMMARY OF THE INVENTION

In a first aspect, the present invention provides a method for producing coffee beans, comprising:
a) providing a plant material comprising coffee cherries,
b) inoculating the plant material using Pichia kluyveri starter culture,
c) inoculating the plant material using Lactiplantibacillus plantarum starter culture,
d) fermenting the plant material to obtain fermented coffee material, and
e) processing the fermented coffee material to obtain coffee beans,
   wherein step b) and c) are carried out at the same time or separately.

In a second aspect, the present invention provides coffee beans obtained by the methods disclosed herein.

In a third aspect, the present invention provides coffee beans fermented with Pichia kluyveri and Lactiplantibacillus plantarum.

In a fourth aspect, the present invention provides use of Pichia kluyveri and Lactiplantibacillus plantarum starter culture for fermenting coffee cherries.

It has been discovered that fermentation of coffee material with both Pichia kluyveri and Lactiplantibacillus plantarum lead to an improvement of the flavor profile. For example, it has been found that Pichia kluyveri and Lactiplantibacillus plantarum may be used to decrease the levels of off-flavor compounds including acetic acid.

A coffee producer can improve fruity flavor by co-inoculating, simultaneously or non-simultaneously, Pichia kluyveri and Lactiplantibacillus plantarum to a freshly harvested batch of coffee cherries.

Furthermore, controlled fermentation minimizes the risk of spoilage which would otherwise occur during spontaneous fermentation.

The present methods allow for the development of desirable characteristics, such as flavor compounds and other quality assets of the fermented coffee beans and the coffee-based products prepared therefrom.

Another aspect of the invention relates to roasted coffee beans, coffee beverage, or coffee-based products prepared from the produced coffee beans of the present invention.

A further aspect relates to the use of a starter culture composition comprising Pichia kluyveri and Lactiplantibacillus plantarum for fermenting coffee material.

### Definitions

The term “plant material” includes anything that is or was live vegetation, in particular plants and any parts thereof.

The term “fermentation” refers generally to any activity or process involving enzymatic decomposition (digestion) of organic materials by microorganisms. The term “fermentation” encompasses both anaerobic and aerobic processes, as well as processes involving a combination or succession of one or more anaerobic and/or aerobic stages.

A “spontaneous fermentation” as used herein is one that employs microorganisms naturally present in and/or unconsciously introduced into the fermented organic material at the start or during fermentation. In contrast, the present invention provides “controlled fermentation” which involves the deliberate addition microbial strains as described here.

The term “coffee-based products” herein refers to products that are prepared using fermented coffee beans. The products intend to refer to the products prepared starting from coffee beans that have been subjected to fermentation as described herein. Coffee-based products can be in a liquid form or in a dry or lyophilized form such as in the form of granules, pellets or a powder. An example of a coffee-based product could e.g. be coffee as such. Another example could e.g. be a coffee cake.

“Coffee cherry” is the fruit of the coffee plant. They have a berry-like appearance and start as green, small, and unripe fruit. It is in the art sometimes referred to as coffee berry.

A “coffee bean” is a seed of the coffee plant and the source for coffee. It is the pit inside the red or purple coffee cherry. Even though they are seeds, they are often referred to as “beans” because of their resemblance to true beans.

The term “a plant material comprising coffee cherries” is the starting material from which coffee beans are produced. Depending on the processing method used, (e.g. “wet processing” or “dry processing”, etc), there may be removed the skin and pulp of the cherries before start of the fermentation process.

The term “starter culture” refers to a composition (also referred to herein as “starter culture composition”) comprising live microorganisms that are capable of initiating or effecting fermentation. A starter culture as used herein refers to is a high-density culture of microorganisms.

The term “co-inoculating” or “co-inoculation” of Pichia kluyveri and Lactiplantibacillus plantarum should be understood that the inoculation of each microorganism need not be at the same time. It may take place in succession.

In the present context, the term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties (e.g. regarding enhancement of desirable coffee flavor compounds) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the yeast genome have been shifted with another nucleotide, or deleted, compared to the mother strain.

### BRIEF DESCRIPTION OF THE FIGURE

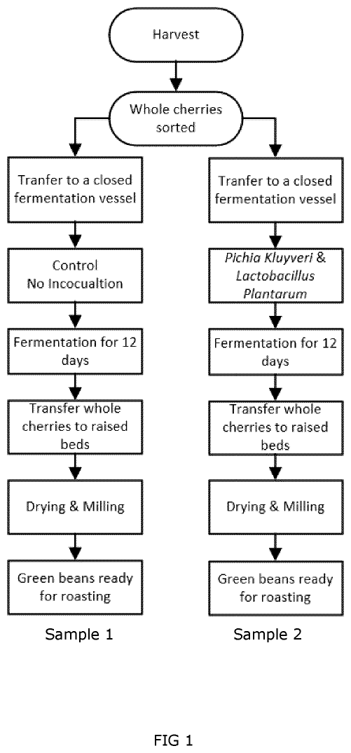

### FIG. 1

FIG. 1 is a schematic representation of the processes used to prepare Sample 1 (control) and Sample 2 (inoculation with both Pichia kluyveri and Lactiplantibacillus plantarum) in Example 1.

### DETAILED DESCRIPTION OF THE INVENTION

Unless defined otherwise, all technical and scientific terms used herein have the same meaning as commonly understood by persons skilled in the art. Although any methods and materials equivalent or similar to those described herein can be used in the practice of the present disclosure, typical methods and materials are described. The detailed information provided for the use of the present invention relates to the methods of the invention as well.

Coffee bean production in general is known. Relevant steps such as e.g. washing, pulping, drying, roasting of the coffee beans may be performed according to standard known processes for making coffee. Accordingly, herein it is not necessary to describe such standard processes in great details.

The first aspect of the invention relates to a method for producing coffee beans comprising the steps of
providing a plant material comprising coffee cherries,
inoculating the plant material using Pichia kluyveri starter culture,
inoculating the plant material using Lactiplantibacillus plantarum starter culture,
fermenting the plant material to obtain fermented coffee material, and
processing the fermented coffee material to obtain coffee beans, wherein the inoculation of P. kluyveri and L. plantarum is carried out at the same time or separately.

The present application is characterized in the combined use of Pichia kluyveri and Lactiplantibacillus plantarum. It has been found that such combination is highly advantageous compared to using either microorganism alone.

To carry out the present invention, a plant material comprising coffee cherries is provided. The plant material is co-inoculated with Pichia kluyveri and Lactiplantibacillus plantarum using starter cultures of the microorganisms. These microorganisms are added to the plant material in an amount which is sufficient to initiate and maintain fermentation. In general, a skilled person is able to determine suitable concentrations of inoculation using routine methods and in view of the current description and examples.

Preferably, the plant material is inoculated with Pichia kluyveri starter culture at a concentration of at least 10² CFU/g, such as at least 10³ CFU/g, at least 10⁴ CFU/g, or at least 10⁵ CFU/g plant material.

Preferably, the plant material is inoculated with Lactiplantibacillus plantarum starter culture at a concentration of at least 10² CFU/g, such as at least 10³ CFU/g, at least 10⁴ CFU/g, or at least 10⁵ CFU/g plant material.

The plant material preferably consists essentially of coffee cherries, which can be whole cherry or pulped cherry. Preferably, the amount of the plant material is at least 50 kg, more preferably at least 100 kg, such as at least 200 kg, such as at least 500 kg or at least 1000 kg.

A preferred embodiment of the invention involves “wet processing” of coffee cherries. In this, the plant material is soaked in water and the starter culture is applied.

Another preferred embodiment of the invention involves “dry processing” of coffee cherries, wherein the plant material is not soaked in water prior to inoculation.

Furthermore, in a preferred embodiment, when using the “natural processing” method, it may be preferred that the inoculation of the starter cultures is done by spraying the culture out over the plant material spread out to dry.

The inoculation using P. kluyveri and L. plantarum starter cultures is carried out at the same time or separately. For example, inoculation with P. kluyveri can take place before inoculation with L. plantarum or vice versa.

The inoculation may be done using any routine methods known in the art. Preferably, the starter culture is applied by spraying a suspension of the starter culture(s).

In preferred embodiments, the concentration of viable cells in the suspension is in the range of 10³ to 10¹² cfu (colony forming units) per ml of the composition, including at least 10⁴ cfu per ml of the composition, such as at least 10⁵ cfu/ml, e.g. at least 10⁶ cfu/ml, such as at least 10⁷ cfu/ml, e.g. at least 10⁸ cfu/ml, such as at least 10⁹ cfu/ml, e.g. at least 10¹⁰ cfu/ml, such as at least 10¹¹ cfu/ml, such as at least 10¹² cfu/ml.

In preferred embodiments, the present application provides a method for producing coffee beans comprising the steps of:
a) providing a plant material comprising coffee cherries,
b) inoculating the plant material using Pichia kluyveri starter culture at a concentration of at least 10² CFU/g plant material
c) inoculating the plant material using Lactiplantibacillus plantarum starter culture at a concentration of at least 10² CFU/g plant material
d) fermenting the plant material to obtain fermented coffee material, and
e) processing the fermented coffee material to obtain coffee beans,
wherein step b) and c) are carried out at the same time or separately.

In another preferred embodiment, the present application provides a method for producing coffee beans comprising the steps of:
a) providing a plant material comprising coffee cherries,
b) inoculating the plant material using Pichia kluyveri starter culture at a concentration of at least 10³ CFU/g plant material
c) inoculating the plant material using Lactiplantibacillus plantarum starter culture at a concentration of at least 10³ CFU/g plant material
d) fermenting the plant material to obtain fermented coffee material, and
e) processing the fermented coffee material to obtain coffee beans,
wherein step b) and c) are carried out at the same time or separately.

The inoculated plant material is then subjected to a suitable condition and the fermentation process begins and continues for a period of time. A person of ordinary skill in the art knows how to select suitable process conditions. Preferably, the fermentation is at least 12 hours, such as at least 12 hours over night, such as at least 1 day, such as at least 2 days, at least 3 days, including up to 5-10 days.

The fermentation may be performed at ambient temperatures, which can include a temperature from 5° C. to 40° C. Preferably, the fermentation can be carried out at a temperature between 10-30° C. In more preferred embodiments, the fermentation is carried out at a temperature of between 2° and 30° C. The fermentation can be carried out in open or closed vessels, or alternatively in the open on the ground.

During the fermentation process, a skilled person in the art is able to adjust other fermentation parameters known to him in order to achieve the desired end-product.

After fermentation, fermented coffee material can be obtained and then subject to further processing steps to obtain coffee beans.

In preferred embodiments, the Pichia kluyveri starter culture is a high-density culture which contains at least 10⁷ CFU/g Pichia kluyveri yeast cells, such as at least 10⁸ CFU/g, at least 10⁹ CFU/g, at least 10¹⁰ CFU/g, at least 10¹¹ CFU/g, and at least 10¹² CFU/g. More than one strains may be present.

In preferred embodiments, the Pichia kluyveri yeast cells can be hydrated or dehydrated. Examples of hydrated cells includes baker's yeast cake, compressed yeast and cream yeast. Examples of dehydrated cells include instant dry yeast, active dry yeast (ADY), freeze dried yeast, and partially dried compressed yeast.

The term “compressed yeast” refers herein to a yeast with a dry matter content of between 35% and 90% (w/w) conventionally produced by propagation of yeast in a fermenter followed by concentration, filtration, extrusion and optionally partial drying on a drier, such as a fluid bed drier. In some embodiments, the dry matter content is between 30% and 45%, such as between 30% and 40% or between 35% and 45%.

The term “cream yeast” herein refers to liquid yeast with a dry matter content of below 28% (w/w) conventionally produced by propagation of yeast in a fermenter followed by concentration by centrifugation.

The term “active dried yeast” or “ADY” refers herein to yeast with a dry matter content of more than 90% (w/w) conventionally produced by propagation of yeast in a fermenter followed by concentration, filtration, extrusion and drying on a fluid bed drier.

The term “partially dried compressed yeast” refers herein to a yeast with a dry matter content of between 45% to 90% (w/w) produced by propagation of yeast in a fermenter followed by concentration, filtration, extrusion and partial drying on a drier, such as a fluid bed drier.

The term “freeze-dried yeast” refers lyophilized cultures in the form of a powder or pellets. In this form, the starter can be shipped without refrigeration.

The starter culture compositions of the present application may additionally comprise cryoprotectants, lyoprotectants, antioxidants, nutrients, fillers, flavorants or mixtures thereof. The composition may be in any form, including liquid (such as slurry), frozen or dried form (such as freeze dried or spray dried). The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including emulsifiers (such as sorbitan monostearate), fillers (such as lactose, maltodextrin) and/or flavorants.

In preferred embodiments, the Lactiplantibacillus plantarum starter culture is a high-density culture which contains at least 10⁷ CFU/g Lactiplantibacillus plantarum bacterial cells, such as at least 10⁸ CFU/g, at least 10⁹ CFU/g, at least 10¹⁰ CFU/g, at least 10¹¹ CFU/g, and at least 10¹² CFU/g. More than one strains may be present.

In one preferred embodiment, the starter culture may be in frozen, liquid or dried form, including e.g. freeze dried form and spray/fluid bed dried form, or frozen or freeze-dried concentrates. The starter cultures may comprise more than one strains yeast or bacteria.

Any suitable Lactiplantibacillus plantarum can be used. In preferred embodiments, the Lactiplantibacillus plantarum is Harvest LB-1 (commercially available from Chr. Hansen A/S) or DSM 27565 as described in WO2015/110484. Mutants thereof having similar properties for the purpose of the present application may also be employed.

Any suitable Pichia kluyveri can be used. In preferred embodiments, the Pichia kluyveri is PK-KR1 or PK-KR2 deposited at the National Measurement Institute, 541-65 Clarke Street, South Melbourne, Victoria 3205, Australia, by University of Auckland, School of Biological Sciences, Auckland 1142, New Zealand, and given the accession numbers V06/022711 and V06/022712, respectively, as described in WO2009/110807. In another preferred embodiments, the Pichia kluyveri is the Pichia kluyveri DSM 28484 as described in WO 2020/035268. Mutants thereof having similar properties for the purpose of the present application may also be employed. In another preferred embodiment, the Pichia kluyveri is the Pichia kluyveri DSM 34278 or DSM 34279.

Preferably, the method as described herein further comprises the step of drying the fermented coffee material obtain dried beans. The dried beans can be stored and transported to other destinations. Preferably, method further comprises the step of roasting the dried beans using any known methods in the art.

Preferably, during fermentation, the plant material is submerged preferably fully in water.

In a preferred embodiment the coffee plant material is Coffea arabica, Coffea canephora Coffea eugenioides or Coffea liberica.

Accordingly, a second aspect of the present invention relates to coffee beans obtained or obtainable by the methods described herein.

A third aspect of the invention relates coffee beans fermented with Lactiplantibacillus plantarum and Pichia kluyveri.

In a preferred embodiment, the coffee beans were fermented with Lactiplantibacillus plantarum (such as Harvest LB-1 or DSM 27565) and Pichia kluyveri (such as DSM 34278, DSM 34279, DSM 28484, PK-KR1 or PK-KR2)

In a further aspect, the present application provides use of Pichia kluyveri and Lactiplantibacillus plantarum for fermenting coffee material. The microorganisms can be used to inoculate plant material by the methods as described in the present application.

In a preferred embodiment the plant material is fermented with the said microorganisms for at least 12 hours, such as for at least 24 hours, such as for at least 2 days, such as for at least 3 days, such as for at least 4 days, such as for at least 5 days, such as for at least 6 days, such as for at least 7 days, such as for at least 8 days, such as for at least 9 days, such as for at least 10 days.

Pichia kluyveri and Lactiplantibacillus plantarum can be in the same or separate starter culture compositions. In some embodiments, the starter culture may be frozen or freeze-dried starter cultures as so-called “Direct Vat Set” (DVS) cultures, intended for direct inoculation into a fermentation vessel or vat. Furthermore, the composition may also be a liquid that is obtained after suspension of the frozen, dried or freeze-dried cell concentrates in a liquid medium such as water or PBS buffer.

In preferred embodiments, the present application provides use of Pichia kluyveri and Lactiplantibacillus plantarum for fermenting coffee material wherein the use is carried out by a method comprising the steps of:
a) providing a plant material comprising coffee cherries,
b) inoculating the plant material using Pichia kluyveri starter culture at a concentration of at least 10² CFU/g plant material
c) inoculating the plant material using Lactiplantibacillus plantarum starter culture at a concentration of at least 10² CFU/g plant material
d) fermenting the plant material to obtain fermented coffee material, and
e) processing the fermented coffee material to obtain coffee beans,
wherein step b) and c) are carried out at the same time or separately.

In another preferred embodiment, the present application provides use of Pichia kluyveri and Lactiplantibacillus plantarum for fermenting coffee material wherein the use is carried out by a method comprising the steps of:
a) providing a plant material comprising coffee cherries,
b) inoculating the plant material using Pichia kluyveri starter culture at a concentration of at least 10³ CFU/g plant material
c) inoculating the plant material using Lactiplantibacillus plantarum starter culture at a concentration of at least 10³ CFU/g plant material
d) fermenting the plant material to obtain fermented coffee material, and
e) processing the fermented coffee material to obtain coffee beans,
wherein step b) and c) are carried out at the same time or separately.

The inventors discovered that the synergism between Pichia kluyveri and Lactiplantibacillus plantarum contributes positively to the flavor profile of coffee products. compared to using Pichia kluyveri or Lactiplantibacillus plantarum alone.

Preferably, the methods as described herein are particularly useful for commercially relevant large-scale production of coffee. The present invention is therefore able to help reduce batch to batch quality inconsistency.

Fermentation with Pichia kluyveri and Lactiplantibacillus plantarum may increase the flavor of the coffee due to the presence of increased amounts of desired flavor compounds.

The method as described herein may also be performed to reduce off-flavors (such as vinegary off flavors). Furthermore, the lack of fruitiness may be overcome acidic profile may be improved.

As discussed above—the results of Example 2 herein demonstrated that coffee beverage prepared from roasted coffee beans fermented with Pichia kluyveri and Lactiplantibacillus plantarum lead to a satisfactory improvement flavor profile compared to control. Sensory improvement may be determined by cupping analysis as set out in Example 2 or other known methods such as according to the Specialty Coffee Association (SCA) cupping protocol widely used (Lingle, T. R. (2011). Systematic guide to the sensory evaluation of Coffee's flavor. In Specialty Coffee Association of America (Ed.), The coffee cupper's handbook (pp. 1-66). Specialty Coffee Association of America). The levels of volatile compounds may be determined by head-space gas chromatography couple with flame ionization detection as set out in Example 3 herein or by any other method known to the skilled person.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Taxonomy

Lactobacillus plantarum is now known as Lactiplantibacillus plantarum as described in Zheng et al., Int. J. Syst. Evol. Microbiol. DOI 10.1099/ijsem.0.004107.

### DEPOSIT AND EXPERT SOLUTION

The applicant requests that a sample of the deposited microorganisms stated below may only be made available to an expert, subject to available provisions governed by Industrial Property Offices of States Party to the Budapest Treaty, until the date on which the patent is granted.

The applicant deposited the DSM 34278 on 31 May 2022 at Leibniz Institute DSMZ-Deutsche Sammlung von Mikroorganismen und Zellkulturen GmbH (DSMZ), Inhoffenstr. 7B, D-38124 Braunschweig, and received the accession No.: DSM 34278.

The applicant deposited the DSM 34279 on 31 May 2022 at Leibniz Institute DSMZ-Deutsche Sammlung von Mikroorganismen und Zellkulturen GmbH (DSMZ), Inhoffenstr. 7B, D-38124 Braunschweig, and received the accession No.: DSM 34279.

The applicant deposited the DSM 28484 on 5 Mar. 2014 at Leibniz Institute DSMZ-Deutsche Sammlung von Mikroorganismen und Zellkulturen GmbH (DSMZ), Inhoffenstr. 7B, D-38124 Braunschweig, and received the accession No.: DSM 28484.

The applicant deposited Lactiplantibacillus plantarum DSM 27565 on 1 Aug. 2013 at Leibniz Institute DSMZ-Deutsche Sammlung von Mikroorganismen und Zellkulturen GmbH (DSMZ), Inhoffenstr. 7B, D-38124 Braunschweig, and received the accession No.: DSM 27565.

### EXAMPLES

### Example 1 Processing of Coffee Beans

The trial was performed on coffee cherries using a natural processing method where a pre-treatment of the cherries was performed by fermented the cherries in vessels without water addition. An uninoculated control batch was included for comparison for both cupping analysis (Example 2) and volatile aroma analysis (Example 3) of the resulting coffee. The two batch were prepared the same way apart from inoculation.

In detail, Coffea arabica cherries from the same harvest day and harvest lot were sorted to remove overripe and unripe cherries, before they were divided into two closed fermentation vessels. The whole cherries were fermented in these two vessels for 12 days.

Batch 1 (control): no inoculation (spontaneous fermentation)

Batch 2: inoculated with Lactiplantibacillus plantarum and Pichia kluyveri

### Starter Cultures

1. Freeze dried Lactiplantibacillus plantarum starter culture DSM 27565 Inoculation rate: 1×10⁷ CFU/g coffee cherry
2. Frozen Pichia kluyveri starter culture PK-KR1 Inoculation rate: 1×10⁶ CFU/g coffee cherry

### Inoculation Protocol

Preparation and inoculation of Lactiplantibacillus plantarum:
Freeze dried starter culture pellets were mixed with 10 times the amount of water (unchlorinated) at room temperature.
The mixture was homogenized with a clean spoon before waiting 5 minutes.
The bacteria suspension was poured into a cleaned watering can and spread over the coffee cherries distributing the equal volume to each of the vessels.

Preparation and inoculation of Pichia kluyveri:
A frozen starter culture was transferred from a freezer to a water bath with lukewarm water which is approximately 30° C. for 1.5 h.
The liquid yeast was splashed as uniformly as possible on top of the coffee cherries in the tank.

Fermentation was carried out for 12 days. The fermented whole cherries were transferred to raised beds for drying.

The two batches were dried to the same degree of moisture content before they were milled and packaged as green coffee beans.

### Example 2 Sensory Evaluation

### 2.1 Material and Methods

Green coffee beans were roasted with the same roasting profile and fresh coffee was prepared from batch 1 and 2 (designated sample 1 and 2, respectively) by grinding 12 g coffee and using 1:17 coffee-to-water ratio at 94-95° C. After 4 minutes the crust is broken and scaped followed by cupping.

In the sensory analysis, attributes of each sample were evaluated through the by cupping analysis according Excellence) to the CoE (Coffee of methodology (http://cupofexcellence.org/rules-protocols/) The following attributes were evaluated: clarity, sweetness, acidity, mouthfeel, flavor, aftertaste, balance and overall. In this methodology, each sample has a starting note of 36 points, to which the scores for each attribute (0-8) are incorporated to make the Final Score.

### 2.2 Results

TABLE 1
Grading results from cupping:

| | | Sample 2 inoculation |
|---|---|---|
| | Sample 1 | with P. kluyveri |
| Attributes | no inoculation | and L. plantarum |
| Clarity | 4 | 6 |
| Sweetness | 5.5 | 6.25 |
| Acidity | 5 | 6.5 |
| Mouthfeel | 4.5 | 6.25 |
| Flavor | 4.5 | 6.5 |
| Aftertaste | 5 | 6 |
| Balance | 5 | 6.25 |
| Overall | 5 | 6.5 |
| Final Score | 74.5 | 86.25 |

TABLE 2
Generic cupping notes:

| | Cupping notes |
|---|---|
| Sample 1 | Baggy, flat, murky flavor, herbal, damp leaves |
| Sample 2 | Hibiscus, red apple, dried spice, raspberry, |
| | sour cherry, grapefruit |

### 2.3 Conclusion

As demonstrated herein, the combined use of Pichia kluyveri and Lactobacillus plantarum advantageously increased the quality grading of the coffee and therefore a higher commercial value of the green beans. A grading above 85 can be considered as having exceptional quality. High quality score has a direct impact on determining specialty coffee prices

### Example 3 VOC Analysis

### 3.1 Material and Methods

Coffee beans from the 2 batches were roasted and brewed the same way as Example 2. Afterwards, 1 L coffee from each sample was strained and frozen for analysis of volatile compounds at Laboratorio de Análisis del Aroma y Enología (LAAE) at the University of Zaragoza, Spain.

### 3.1.1 Analysis of Major Volatile Compounds

Determination of major compounds using the method developed for major volatile compounds based on gas chromatographic-flame ionization detection analysis (GC-FID) of dichloromethane microextracts according to Ortega, Catalina, et al. “Fast analysis of important wine volatile compounds: Development and validation of a new method based on gas chromatographic-flame ionisation detection analysis of dichloromethane microextracts.” Journal of Chromatography A 923.1-2 (2001): 205-214. 3.1.2 Analysis of minor and trace volatile compounds

Determination of minor and trace volatile compounds by solid-phase extraction (SPE) and gas chromatography with mass spectrometric detection (GC-MS) according to Lopez, Ricardo, et al. “Determination of minor and trace volatile compounds in wine by solid-phase extraction and gas chromatography with mass spectrometric detection.” Journal of Chromatography A 966.1-2 (2002): 167-177.

### 3.2 Results

TABLE 3
Level of selected compounds (ppm) detected from Sample 1 and 2.

| | | |
|---|---|---|
| | Sample 1 | |
| Compound | (Control) | Sample 2 |

Carbonyl compounds (oxidative, lactic aromatic descriptors)

| | | | | |
|---|---|---|---|---|
| Acetaldehyde | 2.16 | ppm | 2.60 | ppm |

Acetates (flowery and fruity aromatic descriptors)

| | | | |
|---|---|---|---|
| Ethyl acetate | <DL | 0.04 | ppm |

Acids (cheese aromatic descriptor, except
the vinegar aroma for the acetic acid)

| | | | | |
|---|---|---|---|---|
| Acetic acid | 301 | ppm | 179 | ppm |

Monoterpenols (green, citric aromatic descriptors)

| | | | | |
|---|---|---|---|---|
| α-Terpineol | 2.52 | ppb | 3.14 | ppb |

Phenols (animal, leather aromatic descriptors)

| | | | | |
|---|---|---|---|---|
| 4-Ethylphenol | 10.4 | ppb | 7.88 | ppb |
| 4-Vinylguaiacol | 3204 | ppb | 2277 | ppb |
| 4-Vinylphenol | 3822 | ppb | 3348 | ppb |

### 3.3 Conclusion

The use of the combination of Pichia kluyveri and Lactobacillus plantarum lead an increase in aromatic compounds responsible for positive fruity and fermented flavor descriptors like acetaldehyde, ethyl acetate and α-terpineol. Further, the solution caused a decrease in acetic acid content.

### Example 4 Processing of Coffee Beans

The trial was performed on coffee cherries using a washed processing method where a soaking step was performed on the de-pulped parchment in vessels with minimal water addition. An uninoculated control batch as well as batch inoculated with L. plantarum or P. kluyveri alone was included for comparison for cupping analysis (Example 5) of the resulting coffee. The six batches were prepared the same way apart from inoculation.

In detail, Coffea arabica cherries from the same harvest day and harvest lot were sorted to remove overripe and unripe cherries, before they were mechanically de-pulped. The parchment were fermented/soaked for 48 hours in plastic bins before it was washed and dried.

Batch 1 (control): no inoculation (spontaneous fermentation)

Batch 2: inoculated with Lactiplantibacillus plantarum DSM 27565

Batch 3: inoculated with Pichia kluyveri PK-KR1

Batch 4: inoculated with Pichia kluyveri DSM 28484

Batch 5: inoculated with Lactiplantibacillus plantarum and Pichia kluyveri PK-KR1

Batch 6: inoculated with Lactiplantibacillus plantarum and Pichia kluyveri DSM 28484

Starter cultures
1. Freeze dried Lactiplantibacillus plantarum starter culture DSM 27565
   Inoculation rate: 1×10⁷ CFU/g coffee cherry
2. Frozen Pichia kluyveri starter culture PK-KR1
   Inoculation rate: 1×10⁶ CFU/g coffee cherry
3. Frozen Pichia kluyveri starter culture DSM 28484
   Inoculation rate: 1×10⁶ CFU/g coffee cherry

### Inoculation Protocol

Preparation and inoculation of Lactiplantibacillus plantarum:
Inoculation of Lactiplantibacillus plantarum starter culture were performed by direct addition of freeze dried pellets to the de-pulped parchment and water mixture.

Preparation and inoculation of Pichia kluyveri:
A frozen starter culture was transferred from a freezer to a water bath with lukewarm water which is approximately 30° C. for 1.5 h.
The thawed liquid yeast was splashed as uniformly as possible on top of the coffee cherries in the tank.

All 6 batches were produced simultaneously. All batches went through a mixing step immediately after inoculation.

Fermentation was carried out for 48 hours before all batches were washed and dried to the same degree of moisture content before they were milled and packaged as green coffee beans.

### Example 5 Sensory Evaluation

### 5.1 Material and Methods

In the sensory analysis, coffee was prepared from batch 1-6 (designated sample 1-6) from example 4 and evaluated by cupping analysis according to the SCAA Protocol (Specialty Coffee Association of America Protocols 2015) https://www.scaa.org/PDF/resources/cupping-protocols.pdf.

### 5.2 Results

TABLE 4
Grading results from cupping:

| | | | | | Sample 5 | Sample 6 |
|---|---|---|---|---|---|---|
| | Sample 1 | | | | P. kluyveri | P. kluyveri |
| | no | Sample 2 | Sample 3 | Sample 4 | and | and |
| | inoculation | L. plantarum | P. kluyveri | P. kluyveri | L. plantarum | L. plantarum |
| Aroma | 6 | 6.5 | 6.75 | 6.5 | 6.75 | 7 |
| Flavour | 6 | 7 | 7 | 7 | 7.25 | 7.25 |
| (Notes) | Mold, | Red Grape, | Cooked | Grape, | Cooked | Stewed |
| | Damp, | Paper | Red Fruit, | Popcorn, | Fruit, Plum, | Fruit, Sugar |
| | Earth | | Apple, | Husk | Sweet Grain | Browning |
| | | | Grape, | | | |
| | | | Sweet | | | |
| | | | Grain | | | |
| Aftertaste | 6 | 6.5 | 6.25 | 6.5 | 6.75 | 7 |
| Acidity | 6 | 7 | 7 | 6.75 | 7 | 7 |
| Body | 6 | 6.75 | 7 | 6.5 | 6.75 | 6.75 |
| Balance | 6 | 6.75 | 6.5 | 6.5 | 6.75 | 6.75 |
| Uniformity | 10 | 10 | 10 | 10 | 10 | 10 |
| Clean Cup | 6 | 10 | 10 | 10 | 10 | 10 |
| Sweetness | 10 | 10 | 10 | 10 | 10 | 10 |
| Overall | 6 | 7 | 7 | 6.5 | 7 | 7 |
| Total | 68 | 77.5 | 77.5 | 76.25 | 78.25 | 78.75 |
| Score | | | | | | |

### 5.3 Conclusion

As demonstrated herein, the combined use of Pichia kluyveri and Lactobacillus plantarum advantageously increased the quality grading of the coffee compared to when used alone.

### Example 6 Processing of Coffee Beans

The trial was performed on de-pulped coffee cherries using a processing method known as honey process, where a the cherries were de-pulped after sorting, followed by inoculation by spraying directly on the de-pulped parchmenton on drying beds. An uninoculated control batch as well as batch inoculated with L. plantarum or P. kluyveri alone was included for comparison for cupping analysis (example 7) of the resulting coffee. The four batch were prepared the same way apart from inoculation.

In detail, Coffea arabica cherries from the same harvest day and harvest lot were sorted to remove overripe and unripe cherries, before they were mechanically de-pulped and divided into four smaller batches of equal size and spread out on raises drying beds. The parchment were then dried to 11% moisture content.

Batch 1 (control): no inoculation (spontaneous fermentation)

Batch 2: inoculated with Lactiplantibacillus plantarum Harvest LB-1 Batch 3: inoculated with Lactiplantibacillus plantarum and Pichia kluyveri PK-KR1

Batch 4: inoculated with Lactiplantibacillus plantarum and Pichia kluyveri DSM 28484

### Starter Cultures

1. Freeze dried Lactiplantibacillus plantarum starter culture Harvest LB-1
   Inoculation rate: 1×10⁷ CFU/g coffee cherry
2. Frozen Pichia kluyveri starter culture PK-KR1
   Inoculation rate: 1×10⁶ CFU/g coffee cherry
3. Frozen Pichia kluyveri starter culture DSM 28484
   Inoculation rate: 1×10⁶ CFU/g coffee cherry

### Inoculation Protocol

Preparation and inoculation of Lactiplantibacillus plantarum:
Freeze dried starter culture pellets were mixed with 10 times the amount of water (unchlorinated) at room temperature.
The mixture was homogenized with a clean spoon before waiting 5 minutes.
The bacteria suspension was poured into a cleaned watering spray bottle and sprayed over the coffee cherries distributing the equal volume to each batch of coffee to be inoculated, directly on the raised drying bed.
   Preparation and Inoculation of Pichia kluyveri:
A frozen starter culture was transferred from a freezer to a water bath with lukewarm water which is approximately 30° C. for 1.5 h.
The thawed liquid yeast was poured into a cleaned watering spray bottle and sprayed over the coffee cherries distributing the equal volume to each batch of coffee to be inoculated, directly on the raised drying bed.

The batches of coffee is fermenting in the early stages of the drying process, while water activity still allows for microbial activity. The four batches were dried to the same degree of moisture content (10%) which took approximately 10 days. Thereafter all coffees were milled and packaged as green coffee beans.

### Example 7 Sensory Evaluation

### 7.1 Material and Methods

Coffee was prepared from batch 1-4 (designated sample 1-4) from example 6 and evaluated as described under Example 2.1

### 7.2 Results

TABLE 5
Grading results from cupping:

| | | | Sample 3 | Sample 4 |
|---|---|---|---|---|
| | Sample 1 | | L. plantarum | L. plantarum |
| | no | Sample 2 | and Pichia | and Pichia |
| Attributes | inoculation | L. plantarum | kluyveri | kluyveri |
| Clarity | 6 | 6 | 6 | 7 |
| Sweetness | 6 | 6 | 6.25 | 6.25 |
| Acidity | 6 | 6 | 5.75 | 6.25 |
| Mouthfeel | 6 | 6 | 6 | 6.5 |
| Flavor | 6.75 | 4 | 6 | 6 |
| Aftertaste | 5 | 6 | 6 | 6 |
| Balance | 6 | 6 | 6 | 6 |
| Overall | 5 | 6 | 6 | 6 |
| Final Score | 83.75 | 82 | 84 | 86 |

TABLE 6
Generic cupping notes:

| | Cupping notes |
|---|---|
| Sample 1 | citrus, apricot, jelly like |
| Sample 2 | husky, fenol, hay panela |
| Sample 3 | orange, panela, candly like, apple |
| Sample 4 | strong citrus, candy like |

### 7.3 Conclusion

As demonstrated herein, the combined use of Pichia kluyveri and Lactobacillus plantarum advantageously increased, compared to using either of the microorganism alone. Coffee of very high quality can be obtained.

1. A method for producing coffee beans comprising the steps of:
a) providing a plant material comprising coffee cherries,
b) inoculating the plant material using Pichia kluyveri starter culture,
c) inoculating the plant material using Lactiplantibacillus plantarum starter culture,
d) fermenting the plant material to obtain fermented coffee material, and
e) processing the fermented coffee material to obtain coffee beans,
wherein step b) and c) are carried out at the same time or separately.

2. The method of claim 1, wherein Pichia kluyveri or Lactiplantibacillus plantarum starter culture is inoculated at a concentration of at least 10² CFU/g plant material.

3. The method of claim 1, wherein the inoculating step b) and step c) comprise spraying the starter culture.

4. The method of claim 1, wherein the fermenting of step d) is a fermenting for at least 12 hours.

5. The method of claim 1, wherein the Pichia kluyveri starter culture comprises 10³ to 10¹² CFU/g yeast cells.

6. The method of claim 1, wherein the Lactiplantibacillus plantarum starter culture comprises 10³ to 10¹² CFU/g of bacterial cells.

7. The method of claim 1, wherein the Lactiplantibacillus plantarum is Harvest LB-1, DSM 27565 or mutants thereof.

8. The method of claim 1, wherein, wherein the Pichia kluyveri is DSM 34278, DSM 34279, DSM 28484, PK-KR1, PK-KR2, or mutants thereof.

9. The method of claim 1, wherein step e) comprises drying the fermented coffee material of step d) to obtain dried coffee beans, and optionally roasting the dried coffee beans.

10. The method of claim 1, wherein in step d) the plant material is submerged.

11. The method of claim 1, wherein the coffee plant material is from Coffea arabica, Coffea canephora, Coffea eugenioides or Coffea liberica.

12. Coffee beans obtained by the method according to claim 1.

13. Coffee beans fermented with Pichia kluyveri and Lactiplantibacillus plantarum.

14. (canceled)

15. (canceled)
